SEQUENCE LISTING (6 sequences)

SEQ ID NO: 1 - DNA, 792 bp, synthetic construct
  source 1..792: mol_type = other DNA
atgaagggtggcttcactgggggtgatgagtaccagaagcacttcctgcccagggactacttggctacttactacagcttcgatggcagcccctcacccgaggccgagatgctgaagtttaacttggaatgtctccacaagaccttcggccctggaggcctccaaggggacacgctgattgacattggctcaggtcctaccatctaccaagttcttgctgcctgtgattccttccaagacatcactctctccgactttaccgaccgcaaccgggaggagctggaaaagtggctgaagaaggagccgggggcctatgactggaccccagcggtgaaattcgcctgtgagctggaaggaaacagcggccgatgggaggagaaggaggagaagctgcgggcagcggtgaagcgggtgctcaagtgcgatgtccacctgggcaacccgctggccccggctgtgttgcctctcgccgactgtgtgctcaccctgctggccatggagtgtgcctgctgtagccttgatgcctaccgcgctgccctgtgcaaccttgcctcactgctcaagccgggtggccacctggtgaccactgtcacgcttcggctcccgtcctacatggtggggaagcgtgaattttcctgcgtggccctggagaaagaggaggtggagcaggctgtcctggatgctggctttgacattgaacagctcctacacagtccccagagctactctgtcaccaatgctgccaacaatggggtctgcttcattgtggctcgcaagaagcctgggccctga

SEQ ID NO: 2 - DNA, 1476 bp, synthetic construct
  source 1..1476: mol_type = other DNA
atggcacctctcaccaccatgatcactatactactctcgctccttctagcaggatgcatatactacatcaacgctcgcagagtacggcgctcccacctgccaccaggcccgcctggcatacctatccccttcattgggaatatgtttgatatgccttcagagtctccatggttgacgtttttgcaatggggacgggactatcaaactgatatcctctacgtggatgctgggggatcggagatgattattttgaactcattagaggctataaccgacttgttggaaaagagggggtcgatatactccggtcgacttgagagtacgatggtgaacgagctcatgggatgggagttcgacttgggattcattacctacggcgaaagatggcgcgaagaaaggcgcatgttcgccaaggagttcagcgagaaaaatatccggcaattccgccacgctcaagtgcaagctgccaatcggcttgtcaggcagctcatcaaaacgccaggtcgctggtcgcagcacatccggcatcagatagcggctatgtcgctagatattggttatggaatcgatctcgcagaggatgatccatggcttgaagcaacacagctagcgaacgaagggctcgccatcgcatcagtaccgggcagtttctgggtcgactcatttccctcccttaaataccttccttcttggcttcccggtgctggattcaagcgcaaagcaagggtatggaaagaaggtgccgaccatatggtgaacatgccctatgagacgatgaaaaaactatccgctcaaggcttggcccgaccatcatacgcctcagctcgtcttcaggctatggatcccaacggcgatcttgagcatcaggaacacgtgatcaaaaatacagcgacggaggtcaatgtcggtggcggtgatacgaccgtctctgctatgtcagcgtttattttggctatggtcaaatatcccgaagttcaacgcaaagcccaagcggagctggatatgctcacgagtaaaggccttatcccagattacgacgaggaagatgactcattaccgtatctcacggcatgcgttaaggagctctttcgatggaaccagattgcacccctcgctatcgcgcatcggctcattaaggacgacgtttaccgcgggtatactataccaaagaacgctttggttttcgcgaatacttgggcagtactgaacgatccagaagaatatccagacccctctgagttccgcccagaacggtatctcggtcctgacgggaaacctgaccatacggttcgcgatccccgcaaggcagcattcggctatggtcgacgcacttgccccggactccaccttgcgcagtcgacggtatggattgcaggggcgactcttctgtccgtgttcaatgtcgaacgacctgtggataggactgggaaacctatcgacataccagcggcgtttacgacaggatttttcaggtaa

SEQ ID NO: 3 - DNA, 20 bp, synthetic construct
  source 1..20: mol_type = other DNA
atctcggggcaagaagtgtt

SEQ ID NO: 4 - DNA, 20 bp, synthetic construct
  source 1..20: mol_type = other DNA
acagtggaatggctaaggga

SEQ ID NO: 5 - DNA, 20 bp, synthetic construct
  source 1..20: mol_type = other DNA
tcggatatggcagaaggacc

SEQ ID NO: 6 - DNA, 20 bp, synthetic construct
  source 1..20: mol_type = other DNA
gttgatagcagcaccgtacc

(19) United States
(12) Patent Application Publication
(10) Pub. No.: US 2025/0129399 A1
(43) Pub. Date: Apr. 24, 2025

(54) RECOMBINANT PRODUCTION OF PSILOCIN AND RELATED COMPOUNDS

(71) Applicant: NEUERA PHARMACEUTICALS INC., Markham (CA)

(72) Inventors: Hamed Hasheminejad, Maple (CA); Mahdi Shahsavand, Richmond Hill (CA)

(21) Appl. No.: 18/921,634

(22) Filed: Oct. 21, 2024

Related U.S. Application Data
(60) Provisional application No. 63/591,609, filed on Oct. 19, 2023.

(51) Int. Cl. C12P 17/10; C12N 1/16; C12N 9/02; C12N 9/10; C12N 9/12; C12N 9/22; C12N 15/11; C12N 15/81; C12N 15/90; C12R 1/84

(52) U.S. Cl. CPC C12P 17/10; C12N 1/16; C12N 9/0083; C12N 9/1007; C12N 9/1223; C12N 9/22; C12N 15/111; C12N 15/815; C12N 15/905; C12R 2001/84; C12Y 114/99; C12Y 201/01049

(57) ABSTRACT

Ways of making and using a recombinant organism configured to produce one or more substituted tryptamines, such as psilocin and psilocybin, are provided. The recombinant organism can include a eukaryotic microorganism expressing a recombinant construct. A substituted tryptamine can be produced by a process that includes growing the recombinant organism configured to produce the substituted tryptamine in a growth medium and separating the substituted tryptamine from the recombinant organism and the growth medium. A biosynthetic system for producing a substituted tryptamine is provided that includes a bioreactor, the recombinant organism configured to produce the substituted tryptamine, and a growth medium for the recombinant organism.

### CROSS-REFERENCE TO RELATED APPLICATIONS

This application claims the benefit of U.S. Provisional Application No. 63/591,609 filed on Oct. 19, 2023. The entire disclosure of the above application is incorporated herein by reference.

### FIELD

The present technology relates to the production of psilocin and related compounds, including the biosynthesis of one or more substituted tryptamines using recombinant organisms.

### SEQUENCE LISTING

A sequence listing is submitted electronically herewith and is incorporated herein by reference (filename: 73334-1 Sequence Listing 10-18-2024.xml, date created: Oct. 18, 2024, file size 9 kilobytes). The Sequence Listing submitted is part of the specification and is herein incorporated by reference in its entirety.

### INTRODUCTION

This section provides background information related to the present disclosure which is not necessarily prior art.

Various psychedelic substances are being investigated for therapeutic effects and benefits in relation to various conditions. Examples of psychedelic substances include psilocybin, lysergic acid diethylamide (LSD), and mescaline. The application of psychedelics can promote neurite growth and neuroplasticity and these substances can operate as potent psychoplastogens. It further appears that psychedelics may induce molecular and cellular adaptations related to neuroplasticity and that these effects could be responsible for the various therapeutic benefits associated with these compounds. In particular, psychedelics have shown promise in treating patients with disorders such as obsessive-compulsive disorder (OCD), post-traumatic stress disorder (PTSD), nicotine dependence, alcohol dependence, depression, anxiety disorders, and cluster headaches. Psychedelics have also been shown to have potent anti-inflammatory activity in models of inflammatory diseases including asthma, cardiovascular disease, and diabetes.

The psychedelic substances psilocybin and psilocin belong to a class of compounds referred to as substituted tryptamines or serotonin analogues. The molecular structures of such substituted tryptamines include an indole ring (six-membered benzene ring fused to a five-membered pyrrole ring), joined to an amino group (NH₂) via an ethyl sidechain (—CH₂—CH₂—). In substituted tryptamines, the indole ring, sidechain, and/or amino group are modified by substituting one or more groups for one or more of the hydrogen (H) atoms. Examples of substituted tryptamines include the neurotransmitter serotonin and the hormone melatonin, which is involved in regulating the sleep-wake cycle. Substituted tryptamines also include what are referred to as tryptamine alkaloids, which can be found in fungi, plants, and animals, of which psilocin and psilocybin are examples.

An extensive number of substituted tryptamines are known. Several substituted tryptamines are naturally occurring, and several substituted tryptamines can be synthetically produced. A particular non-exhaustive list of substituted tryptamines includes 4-hydroxytryptamine, norbaeocystin (4-phosphoryl oxy-tryptamine), NMT (N-methyltryptamine), 4-hydroxy-NMT (4-hydroxy-N-methyltryptamine), baeocystin (4-phosphoryloxy-N-methyl-tryptamine), DMT (N,N-dimethyltryptamine), psilocin (4-hydroxy-N,N-dimethyltryptamine), and the aforementioned psilocybin (4-phosphoryloxy-N,N-dimethyltryptamine).

It would be desirable to biosynthetically produce substituted tryptamines, such as psilocybin and related compounds, in order to optimize productivity, control purity, improve scalability, and maximize cost-effectiveness in obtaining and using these compounds.

### SUMMARY

In concordance with the instant disclosure, a method to biosynthetically produce substituted tryptamines, such as psilocybin and related compounds, in order to optimize productivity, control purity, improve scalability, and maximize cost-effectiveness in obtaining and using these compounds, has surprisingly been discovered.

The present technology includes ways of making and using recombinant microorganisms configured to produce substituted tryptamines, such as psilocin, psilocybin, and related compounds. The recombinant microorganism can include a eukaryotic microorganism expressing a recombinant construct including an indolethylamine N-methyltransferase (INMT) and dimethyl tryptamine 4-hydroxylase. A particular example of a recombinant microorganism is Pichia pastoris. A method of producing psilocin is provided that includes the following: catalyzing conversion of tryptamine to N-methyltryptamine using indolethylamine N-methyltransferase; catalyzing the conversion of N-methyltryptamine to N,N-dimethyl tryptamine using indolethylamine N-methyltransferase; and catalyzing the conversion of N,N-dimethyl tryptamine to psilocin using dimethyl tryptamine 4-hydroxylase.

Various ways can be used to incorporate the recombinant construct into the microorganism, including where the recombinant construct is configured as a single recombinant element or multiple recombinant elements. The recombinant construct can also be incorporated in a single event or where different portions of the recombinant construct are incorporated in a single event or multiple events, including multiple sequential events. The recombinant construct can be incorporated into the genomic DNA of the recombinant microorganism, for example, using CRISPR. Other aspects include where the recombinant organism includes a KU70 gene knockout.

Recombinant microorganisms configured to produce substituted tryptamines can be used in various ways. Substituted tryptamines can be produced by a process that includes growing the recombinant microorganism configured to produce the substituted tryptamine in a growth medium and separating the substituted tryptamine from the recombinant microorganism and the growth medium. Similarly, a biosynthetic system for producing a substituted tryptamine can be provided that includes a bioreactor, the recombinant microorganism configured to produce the substituted tryptamine, and a growth medium for the recombinant microorganism.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 depicts the biosynthesis pathways of certain substituted tryptamines, including psilocybin.

FIG. 2 depicts colony DNA verification for INMT genes.

FIG. 3 depicts INMT protein verification in different time courses.

FIG. 4 depicts HACS11 (transcription factor) plasmid verification.

FIG. 5 depicts PsiH plasmids verification for CRISPR knock-in.

FIG. 6 depicts plasmid verification for INMT PsiH genes.

FIG. 7 depicts DNA verification for INMT genes.

FIG. 8 depicts PsiH-transformed colonies after the CRISPR technique.

FIG. 9 depicts PsiH DNA verification in colonies after the CRISPR technique.

FIG. 10 depicts CRISPR colonies for INMT genes.

FIG. 11 depicts protein verification for INMT and PsiH genes.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

There are several issues with current ways used to produce psychedelics, including certain substituted tryptamines, such as psilocybin and psilocin-related compounds. Preparation of psychedelics for pharmaceutical use and research typically relies upon chemical synthesis through difficult and costly processes. Although the chemical synthesis of psilocybin has improved since its initial discovery in 1959 by Hoffman et al., who achieved final yields of 20% semi-pure psilocybin, it continues to challenge chemists due to both the difficulty of certain synthetic steps, the phosphorylation of psilocin, as well as the stereospecific 4-hydroxylation of the indole aromatic ring. Recently, the company Compass Pathways developed and patented a new method for the chemical synthesis of psilocybin (see U.S. Pat. No. 10,519,175 to Londesbrough et al. and related family members), and while this method improves on previous methods with an overall yield of 75%, the method relies upon expensive 4-hydroxyindole as a starting substrate resulting in high production costs which may limit its application.

Biotechnological production of psilocybin and psilocin provides an attractive alternative as it offers the high yields and purity required for pharmaceutical applications, while allowing for a simple and cost-effective production process from a simple and inexpensive starting substrate, such as glucose. Furthermore, unlike chemical synthesis, microbial cell factories can be rapidly adapted to produce a variety of valuable tryptamine derivatives.

A substituted tryptamine precursor of psilocybin is psilocin. Psilocin is an alkaloid tryptamine-derived compound produced mainly by mushrooms from the basidiomycete genus called Psilocybe, also generally known as magic mushrooms. Psilocin is structurally similar to human neurotransmitters such as serotonin. It is therefore not surprising that psilocin has been shown to bind to over fifteen human serotonin-related receptors. Psilocin and psilocybin were classified as Schedule 1 drugs in the USA in 1970 due to their popularity as recreational drugs. Psilocin and psilocybin have seen success in clinical trials for the treatment of different psychological and neurological diseases, including addiction, anxiety, and depression (See Bogensuchutz et al. 2015, Grobetal 2011, and Carhart-Harris et al. 2018).

The natural content of psilocin and psilocybin in psychedelic mushrooms is often quite low. Furthermore, a large variation can be observed between different batches. (Bigwood and Huge 1982). Due to the low existence of natural sources and strenuous isolation methods, natural substituted tryptamines products can be hard to attain in desired quantities.

Psilocin and psilocybin can be synthesized chemically. However, chemical synthesis can be an expensive process. (Nicholas and Frescas 1999). Recently the company Compass Pathways developed a new method for the chemical synthesis of psilocin. However, this method uses 4-hydroxy-indole as a starting substitute resulting in high production costs.

Biotechnology production of psilocin (psilocin biosynthesis) is an alternative that offers a high yield and purity. Also, psilocin biosynthesis can be less costly than chemical synthesis methods. The psilocin biosynthesis pathway was discovered by Trick et al. in 2017. Certain biosynthetic methods for producing psilocin employ the regular biosynthesis pathways and genes found in the psilocybe species. The biosynthetic methods provided herein, however, use another pathway and enzyme to produce psilocin and DMT in Pichia pastoris. The present technology therefore provides a new way to biosynthesize psilocin and related substituted tryptamines.

The psilocin biosynthesis pathway provided by the present disclosure can include the following aspects. A new biosynthetic pathway is introduced instead of using the regular pathway using an enzyme to convert tryptamine to a substituted tryptamine compound, the enzyme known as indolethylamine N-methyltransferase (INMT), also known as amine N-methyltransferase. The INMT enzyme catalyzes the N-methylation of tryptamine and structurally related compounds. In the cortex of the human brain, endogenous INMT enzyme converts tryptamine to N-methyltryptamine (NMT), and then NMT is converted to N,N-dimethyltryptamine (DMT) by another molecule of INMT enzyme. Next, in the biosynthetic pathway provided herein, the DMT can be converted to psilocin using the enzyme dimethyl tryptamine 4-hydroxylase, also known as N,N-dimethyl tryptamine 4-hydroxylase. In certain embodiments, the INMT enzyme can be based on INMT originating from the brain cortex of humans and the dimethyl tryptamine 4-hydroxylase can be based on dimethyl tryptamine 4-hydroxylase originating from Psilocybe cyanescens. Genes for the INMT enzyme and the dimethyl tryptamine 4-hydroxylase can be codon optimized for expression in Pichia pastoris. The INMT gene sequence is shown as follows (SEQ. ID. NO. 1):

ATGAAGGGTGGCTTCACTGGGGGTGATGAGTACCAGAAGCACTTCCTGCC
CAGGGACTACTTGGCTACTTACTACAGCTTCGATGGCAGCCCCTCACCCG
AGGCCGAGATGCTGAAGTTTAACTTGGAATGTCTCCACAAGACCTTCGGC
CCTGGAGGCCTCCAAGGGGACACGCTGATTGACATTGGCTCAGGTCCTAC
CATCTACCAAGTTCTTGCTGCCTGTGATTCCTTCCAAGACATCACTCTCT
CCGACTTTACCGACCGCAACCGGGAGGAGCTGGAAAAGTGGCTGAAGAAG
GAGCCGGGGGCCTATGACTGGACCCCAGCGGTGAAATTCGCCTGTGAGCT
GGAAGGAAACAGCGGCCGATGGGAGGAGAAGGAGGAGAAGCTGCGGGCAG
CGGTGAAGCGGGTGCTCAAGTGCGATGTCCACCTGGGCAACCCGCTGGCC
CCGGCTGTGTTGCCTCTCGCCGACTGTGTGCTCACCCTGCTGGCCATGGA
GTGTGCCTGCTGTAGCCTTGATGCCTACCGCGCTGCCCTGTGCAACCTTG
CCTCACTGCTCAAGCCGGGTGGCCACCTGGTGACCACTGTCACGCTTCGG
CTCCCGTCCTACATGGTGGGGAAGCGTGAATTTTCCTGCGTGGCCCTGGA
GAAAGAGGAGGTGGAGCAGGCTGTCCTGGATGCTGGCTTTGACATTGAAC
AGCTCCTACACAGTCCCCAGAGCTACTCTGTCACCAATGCTGCCAACAAT
GGGGTCTGCTTCATTGTGGCTCGCAAGAAG

The present technology provides a recombinant organism configured to produce one or more substituted tryptamines, where the recombinant organism can be used in certain processes and systems to biosynthetically produce certain substituted tryptamines in order to optimize productivity, control purity, improve scalability, and maximize cost-effectiveness in obtaining and using the substituted tryptamines. A eukaryotic microorganism is provided that can express one or more recombinant constructs including indolethylamine N-methyltransferase (INMT) and dimethyl tryptamine 4-hydroxylase. The eukaryotic microorganism can include a member of the fungus kingdom, such as a methylotrophic yeast, where a particular example includes Pichia pastoris.

The term eukaryotic microorganism, as used herein, can be defined as microscopic organisms whose cells contain a membrane-bound nucleus and other membrane-bound organelles. More specifically, eukaryotic microorganisms include single-celled organisms having a well-defined nucleus where the genetic material (DNA) can be housed. The organelles include, for example, mitochondria, for energy production, or endoplasmic reticulum for protein synthesis. Examples of eukaryotic microorganisms include, but are not limited to, animals, plants, fungi, and protists.

The term recombinant construct, as used herein, can be defined to describe a microorganism having its genetic material artificially altered to include DNA from another organism or altered in a way not found in wild-type examples of the microorganism. Further, the recombinant construct can be defined as a joining of DNA molecules from two different sources or species where the recombined DNA molecule can be inserted into a host organism to produce new genetic combinations. Recombinant constructs can also be defined as a technology used to create new genes with new functions, or to alter genetic material to give living organisms desired characteristics. Further, the term recombinant element can be used to describe a DNA, protein, cell, or organism that has been created by combining genetic material from two or more different sources in order to alter their characterization.

The terms “single event” and “single recombinant event” as used herein can be defined as a piece of DNA that has undergone a single recombination event. More specifically, this means that the DNA has exchanged genetic material with another DNA molecule at only one specific location, resulting in a new combination of genetic information within that DNA segment. The terms “single event” and “single recombinant event” can refer to the alteration of the DNA by a single crossover event during genetic recombination. The terms “multiple events” or “multiple recombinant events” as used herein can be defined as a genetic event that occurs when multiple recombinant events occur in a genome.

The INMT enzyme can include the following aspects. The INMT is an enzyme that can play a role in the biosynthesis of certain substituted tryptamines like psilocin. The INMT enzyme specifically catalyzes the N-methylation of tryptamine and structurally related compounds. Particularly, the INMT enzyme can transfer methyl groups to amino groups of small molecule acceptor compounds. The INMT enzyme is associated with the development and activity of the nervous system and participates in the detoxification of selenium compounds. In the present technology, the INMT can be derived from indolethylamine N-methyltransferase (INMT) expressed in human brain cortex.

The dimethyl tryptamine 4-hydroxylase can include the following aspects. In the present technology, the dimethyl tryptamine 4-hydroxylase can be derived from dimethyl tryptamine 4-hydroxylase originating from Psilocybe cyanescens. It should be appreciated that the dimethyl tryptamine 4-hydroxylase can be used to convert dimethyltryptamine (DMT) to psilocin, as described herein. The DMY hydroxylase gene is as follows (SEQ. ID. NO. 2):

ATGGCACCTCTCACCACCATGATCACTATACTACTCTCGCTCCTTCTAGC
AGGATGCATATACTACATCAACGCTCGCAGAGTACGGCGCTCCCACCTGC
CACCAGGCCCGCCTGGCATACCTATCCCCTTCATTGGGAATATGTTTGAT
ATGCCTTCAGAGTCTCCATGGTTGACGTTTTTGCAATGGGGACGGGACTA
TCAAACTGATATCCTCTACGTGGATGCTGGGGGATCGGAGATGATTATTT
TGAACTCATTAGAGGCTATAACCGACTTGTTGGAAAAGAGGGGGTCGATA
TACTCCGGTCGACTTGAGAGTACGATGGTGAACGAGCTCATGGGATGGGA
GTTCGACTTGGGATTCATTACCTACGGCGAAAGATGGCGCGAAGAAAGGC
GCATGTTCGCCAAGGAGTTCAGCGAGAAAAATATCCGGCAATTCCGCCAC
GCTCAAGTGCAAGCTGCCAATCGGCTTGTCAGGCAGCTCATCAAAACGCC
AGGTCGCTGGTCGCAGCACATCCGGCATCAGATAGCGGCTATGTCGCTAG
ATATTGGTTATGGAATCGATCTCGCAGAGGATGATCCATGGCTTGAAGCA
ACACAGCTAGCGAACGAAGGGCTCGCCATCGCATCAGTACCGGGCAGTTT
CTGGGTCGACTCATTTCCCTCCCTTAAATACCTTCCTTCTTGGCTTCCCG
GTGCTGGATTCAAGCGCAAAGCAAGGGTATGGAAAGAAGGTGCCGACCAT
ATGGTGAACATGCCCTATGAGACGATGAAAAAACTATCCGCTCAAGGCTT
GGCCCGACCATCATACGCCTCAGCTCGTCTTCAGGCTATGGATCCCAACG
GCGATCTTGAGCATCAGGAACACGTGATCAAAAATACAGCGACGGAGGTC
AATGTCGGTGGCGGTGATACGACCGTCTCTGCTATGTCAGCGTTTATTTT
GGCTATGGTCAAATATCCCGAAGTTCAACGCAAAGCCCAAGCGGAGCTGG
ATATGCTCACGAGTAAAGGCCTTATCCCAGATTACGACGAGGAAGATGAC
TCATTACCGTATCTCACGGCATGCGTTAAGGAGCTCTTTCGATGGAACCA
GATTGCACCCCTCGCTATCGCGCATCGGCTCATTAAGGACGACGTTTACC
GCGGGTATACTATACCAAAGAACGCTTTGGTTTTCGCGAATACTTGGGCA
GTACTGAACGATCCAGAAGAATATCCAGACCCCTCTGAGTTCCGCCCAGA
ACGGTATCTCGGTCCTGACGGGAAACCTGACCATACGGTTCGCGATCCCC
GCAAGGCAGCATTCGGCTATGGTCGACGCACTTGCCCCGGACTCCACCTT
GCGCAGTCGACGGTATGGATTGCAGGGGCGACTCTTCTGTCCGTGTTCAA
TGTCGAACGACCTGTGGATAGGACTGGGAAACCTATCGACATACCAGCGG
CGTTTACGACAGGATTTTTCAGGTAA

The recombinant construct can include the following aspects. The recombinant construct can be incorporated into the genomic DNA of the recombinant organism, such as by using Clustered Regularly Interspaced Short Palindromic Repeats (CRISPR), for example. The term CRISPR, as used herein, can be defined as a gene-editing technology allowing modification of the DNA of an organism. The recombinant microorganism can include a KU70 gene knockout. The term gene knockout, as used herein, can be defined as a genetic engineering technique that permanently militates against a specific gene from being expressed in an organism. More specifically, the gene knockout can be referred to as the use of genetic engineering to inactivate or remove one or more specific genes from an organism. The KU70 gene knockout technique, as used herein, can be defined as a technique that permanently removes the expression of the KU70 gene in a cell or organism. As used in the present technology, the KU70 gene knockout can be used for disruption of the KU70 gene to enable high frequency homologous recombination in a eukaryotic recombinant organism.

Substituted tryptamines and ways of producing such can include a process of growing the recombinant microorganism configured to produce the substituted tryptamine in a growth medium and separating the substituted tryptamine from the recombinant microorganism and the growth medium. A biosynthetic system for producing the substituted tryptamine can include a bioreactor, the recombinant microorganism configured to produce the substituted tryptamine, and a growth medium for the recombinant organism.

The term bioreactor, as used herein, can be defined as a vessel in which a biological reaction or change takes place. More specifically, the term bioreactor can be defined as a controlled environment, typically a vessel, designed to support the growth and activity of living organisms like bacteria, yeast, plant or animal cells, allowing them to produce a desired product through a biological process, while also maintaining optimal conditions such as temperature, pH, and nutrient levels for maximum yield. The bioreactor can provide a system where cells or enzymes can carry out chemical reactions in a controlled manner to produce specific substances.

The term growth medium, as used herein, can be defined as a substance or substances that support the growth of microorganisms, cells, or small plants. The term growth medium can further be known as a culture medium, which is a liquid, solid, or semi-solid substance that supports the growth of cells or microorganisms.

The biosynthetic production of substituted tryptamines provided by the present technology realizes several benefits and advantages over the isolation of substituted tryptamines from natural sources, such as fungi from the genus Psilocybe, including the species P. azurescens, P. semilanceata, and P. cyanescens, among others. These benefits and advantages include at least the following: (1) access to psilocybin and related compounds that are currently not economically feasible to extract from fungal sources and develop into drug candidates; (2) cost-savings relative to existing agricultural production methods (e.g., plant-grow-harvest-extract-purify); (3) increased yield of rare substituted tryptamines with optimized fermentation, purification consistency, and quality control; (4) scalability to allow efficient and cost-effective supply as market demand increases; (5) the present technology can produce bio-identical substituted tryptamines to those found in nature; and (6) biosynthesis approaches provide a long-term sustainable option, where a bioreactor has a small footprint relative to the are required for growing fungi and where bioreactor growth can occur in a short time (e.g., days) instead of time necessary to grow fungi, which can also take weeks to several months. The biosynthetic production of substituted tryptamines can further include genes originating from different microorganisms that allow the formation and tailoring of biosynthetic pathways not existing in nature. The present technology can accordingly attain economical production of substituted tryptamines in a bioreactor.

The biosynthesis of various types of substituted tryptamines is contemplated herein. Examples of such substituted tryptamines include 4-hydroxytryptamine, norbaeocystin (4-phosphoryloxy-tryptamine), NMT (N-methyltryptamine), 4-hydroxy-NMT (4-hydroxy-N-methyltryptamine), baeocystin (4-phosphoryloxy-N-methyl-tryptamine), DMT (N,N-dimethyltryptamine), psilocin (4-hydroxy-N,N-dimethyltryptamine), and psilocybin (4-phosphoryloxy-N,N-dimethyltryptamine).

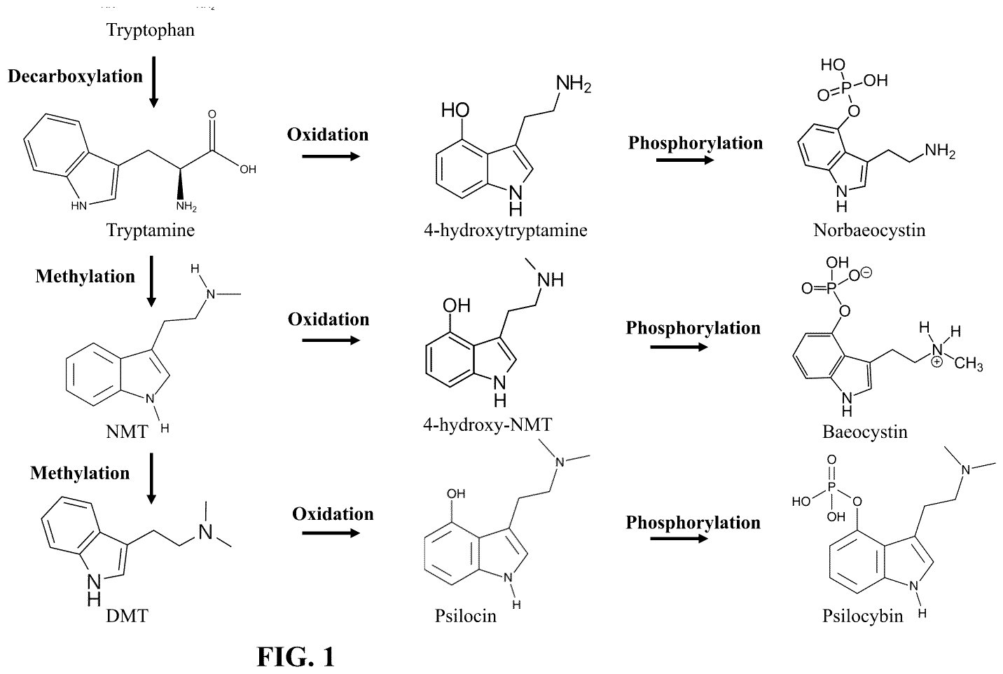

The substituted tryptamine 4-hydroxytryptamine, shown in FIG. 1, can include the following aspects. The 4-hydroxytryptamine is a member of the class of tryptamines that is tryptamine in which the indole ring has been substituted by a hydroxy group. The 4-hydroxytryptamine is a member of tryptamines, a member of hydroxyindoles, and a primary amino compound. The 4-hydroxytryptamine is further a conjugate base of 4-hydroxytryptamine (1+). As shown in FIG. 1, the 4-hydroxytryptamine can be formed by the oxidation of tryptamine.

The substituted tryptamine norbaeocystin, shown in FIG. 1, can include the following aspects. The norbaeocystin is a tryptamine alkaloid that is tryptamine carrying an additional phosphoryloxy substituent. The norbacocystin has a role as a fungal metabolite and a hallucinogen. The norbaeocystin further is an organic phosphate, a tryptamine alkaloid, and a primary amino compound. The norbaeocystin is functionally related to a tryptamine and is a conjugate acid of a norbaeocystin(1−). As shown in FIG. 1, the norbaeocystin can be formed by the phosphorylation of 4-hydroxytryptamine.

The substituted tryptamine NMT, as shown in FIG. 1, can include the following aspects. The NMT is a tryptamine alkaloid and a member of tryptamines. The NMT includes a role as a metabolite. The NMT further is functionally related to a tryptamine and is a conjugate base of a N-methyltryptaminium. As shown in FIG. 1, the NMT can be formed by the methylation of tryptamine.

The substituted tryptamine 4-hydroxy-NMT, as shown in FIG. 1, can include the following aspects. The 4-hydroxy-NMT can also be known as norpsilocin. The 4-hydroxy-NMT is a tryptamine alkaloid. The 4-hydroxy-NMT is known to be a dephosphorylated metabolite of baeocystin. As shown in FIG. 1, the 4-hydroxy NMT can be formed by the oxidation of NMT.

The substituted tryptamine baeocystin, as shown in FIG. 1, can include the following aspects. The baeocystin is a tryptamine alkaloid that is N-methyltryptamine carrying an additional phosphoryloxy substituent. The baeocystin has a role as a hallucinogen and a fungal metabolite. The baeocystin is an organic phosphate, a tryptamine alkaloid, and a secondary amino compound. The baeocystin further is functionally related to a tryptamine and is a conjugate acid of baeocystin(1−). As shown in FIG. 1, the baeocystin can be formed by the phosphorylation of 4-hydroxy-NMT.

The substituted tryptamine DMT, as shown in FIG. 1, can include the following aspects. The DMT is a tryptamine derivative having two N-methyl substituents on the side-chain. The DMT is a tryptamine alkaloid and a member of tryptamines. Further, the DMT is functionally related to a tryptamine. The DMT is a conjugate base of a N,N-dimethyltryptaminium. As shown in FIG. 1, the DMT can be formed by the methylation of NMT.

The substituted tryptamine psilocin, as shown in FIG. 1, can include the following aspects. The psilocin is a tryptamine alkaloid that is N,N-dimethyltryptamine carrying an additional hydroxy substituent. The psilocin is further a hallucinogenic alkaloid that can be isolated in trace amounts from psilocybe mushrooms. The psilocin has a role as a hallucinogen, a serotonergic agonist, a fungal metabolite, a human xenobiotic metabolite, and a drug metabolite. The psilocin is further a tryptamine alkaloid, a tertiary amino compound, a member of hydroxyindoles, and a member of phenols. The psilocin is functionally related to a N,N-dimethyltryptamine and is a conjugate base of a psilocinium. As shown in FIG. 1, the psilocin can be formed by the oxidation of DMT.

The substituted tryptamine psilocybin, as shown in FIG. 1, can include the following aspects. The psilocybin is a tryptamine alkaloid that is N,N-dimethyltryptamine carrying an additional phosphoryloxy substituent. The psilocybin is a major hallucinogenic alkaloid isolated from psilocybe mushrooms. The psilocybin has a role as a hallucinogen, a fungal metabolite, a prodrug, and a serotonergic agonist. The psilocybin is a tryptamine alkaloid, a tertiary amino compound, and an organic phosphate. The psilocybin is further functionally related to a psilocin and is a conjugate acid of psilocybin(1−). As shown in FIG. 1, the psilocybin can be formed by the phosphorylation of psilocin.

The present technology can employ Pichia pastoris as a platform to produce substituted tryptamines. Pichia pastoris is a methylotrophic yeast, a heterotroph discovered in the 1960s. P. pastoris can survive and reproduce using different carbon sources, such as methanol, glucose, and glycerol. P. pastoris exists in two cell types, haploid and diploid cells, obtained through mitosis, sporulation, and meiosis, respectively. P. pastoris is a single-celled eukaryote. Expression of proteins within P. pastoris can be studied and compared with other more complex eukaryotic species to understand their function and origin.

The P. pastoris provides an advanced heterologous gene expression system and can be used to produce various heterologous proteins. Due to its ability to serve as a model organism for genetic studies and as a protein expression system, P. pastoris has become an essential microbial strain for biological research and applications. For example, P. pastoris can functionally process large molecular weight proteins, which is useful in translation hosts.

Particular advantages of P. pastoris fermentation as used in the present technology can include at least the following aspects:
(1) Culture: P. pastoris can be grown on simple, inexpensive media with fast growth rates. P. pastoris is a methylotrophic yeast, where it can be grown with simple methanol as the sole energy source. P. pastoris can also be grown in shake flasks or fermenters, making it suitable for small-scale and large-scale production.
(2) Growth: P. pastoris, like other widely used yeast models, has a relatively short lifespan and fast regeneration time. P. pastoris can be grown in medium with high cell densities. This feature is compatible with the expression of heterologous proteins and allows the production of higher yields.
(3) Production: The P. pastoris expression system is capable of producing high concentrations of heterologous proteins. P. pastoris is easy to manipulate genetically, and a wide range of vectors and strains are available. This eukaryote also has the potential to produce soluble and correctly folded recombinant proteins with post-translational modification (PTM), such as N-glycosylation. The target protein function may require eukaryotic processing that is not available in prokaryotes.

Based on the characteristics of P. pastoris, it has been successfully used to produce a large number of biopharmaceuticals and industrial enzymes. In the pharmaceutical industry, high cell density fermentation of P. pastoris enables the production of large amounts of highly active and low-cost recombinant proteins. In addition, P. pastoris is capable of protein glycosylation for biotherapeutics. In the food industry, P. pastoris is used to produce different kinds of enzymes as food additives with many functions.

The present technology can employ various techniques to engineer and form the recombinant microorganism configured to produce one or more substituted tryptamines. One such methodology includes CRISPR technology for genome editing. Within a few years, genome engineering by CRISPR/Cas9 (clustered regularly interspaced short palindromic repeats/CRISPR associated protein 9) has become a valuable tool in gene editing (Sander & Joung 2014; Wang et al. 2016; Doudna & Charpentier 2014). CRISPR facilitates targeted, programmable genome modifications and provides major advantages over classical knock-out, knock-in approaches as well as alternative genome engineering strategies. Besides its potency in basic research for studying disease conditions (Xue et al. 2014; Matano et al. 2015; Wu et al. 2013; Baltimore et al. 2015), CRISPR/Cas9 is also used for the host (strain) engineering for biotechnological production processes (Roointan & Morowvat 2016; Krappmann 2016; Shen et al. 2017). Prior to CRISPR/Cas9, highly efficient genome manipulations were difficult to achieve in many microorganisms. Gene targeting with conventional knock-out/knock-in cassettes containing homologous sequences depended on the microorganism's amenability for homologous recombination and homology cassettes were predominantly integrated ectopically (Park et al. 2016; Weninger, Killinger, et al. 2015). In addition, the replacement of DNA regions or the insertion of markers (or other disruption cassettes) can change the context of the genome sequence and can influence neighbouring genes and their expression. This can be relevant for maintaining the functional fidelity of tightly packed microbial genomes, where adjacent open reading frames are sometimes only separated by very short DNA stretches.

Genome engineering methods available prior to the advent of CRISPR/Cas9 such as Zinc-finger nucleases or TALENs (Gaj et al. 2013; Kim & Kim 2014; Weninger, Killinger, et al. 2015) required laborious protein engineering to reprogram the targeting locus. CRISPR/Cas9 allows to easily introduce targeted strand breaks in almost any desired genomic locus and can easily be reprogrammed. Paper 3 (expanding CRISPR) Komagataella phaffii (syn. Pichia pastoris) is one of the most commonly used host systems for recombinant protein expression. Achieving targeted genetic modifications had been hindered by low frequencies of homologous recombination (HR). Recently, a CRISPR/Cas9 genome editing system has been implemented for P. pastoris enabling gene knockouts based on indels (insertion, deletions) via nonhomologous end joining (NHEJ) at near 100% efficiency. However, specifically integrating homologous donor cassettes via HR for replacement studies had proven difficult resulting at most in ˜20% correct integration using CRISPR/Cas9.

In the present technology, CRISPR/Cas9 mediated integration of markerless donor cassettes can be achieved at efficiencies approaching 70-90% using a Ku70 deletion strain. The Ku70 gene is involved in NHEJ repair and the lack of the protein appears to favor repair via HR nearly exclusively. While the absolute number of transformants in the AKu70 strain can be reduced, virtually all surviving transformants show correct integration. In the wildtype strain, markerless donor cassette integration can also be improved up to 25-fold by placing an autonomously replicating sequence (ARS) on the donor cassette. Alternative strategies for improving donor cassette integration using a Cas9 nickase variant or reducing off-targeting associated toxicity using a high fidelity Cas9 variant did not produce the desired effects in P. pastoris. Furthermore, the present technology employs Cas9/gRNA expression plasmids with a geneticin (G418) resistance marker which provide a versatile tool for marker recycling. These CRISPR-Cas9 tools can be applied for modifying existing production strains and also enable the markerless whole-genome modification studies in P. pastoris.

The present technology, in particular, provides a new pathway to produce one or more substituted tryptamines, such as psilocybin. In previous methods, genes that were involved in certain pathways had to be introduced to produce the substituted tryptamines. In the technology provided herein, genes from different pathways are combined to produce new avenues of biosynthetic production of substituted tryptamines. One such example is using the CRISPR technique to knock in all genes involved in this process. The present technology can provide a method of producing psilocin by catalyzing the conversion of tryptamine to N-methyltryptamine using indolethylamine N-methyltransferase, catalyzing the conversion of N-methyltryptamine to N,N-dimethyltryptamine using indolethylamine N-methyltransferase, and catalyzing the conversion of N,N-dimethyltryptamine to psilocin using dimethyltryptamine 4-hydroxylase. The present technology can also provide a method of making psilocin including growing the recombinant organism configured to produce the substituted tryptamine in a growth medium and separating the substituted tryptamine from the recombinant microorganism and the growth medium.

Bioinformatic aspects related to genes for the biosynthesis of substituted tryptamines include the following details. Upon the recognition of biochemical pathways, the structure of the genes and proteins can be identified and verified and the focus can be on post-translational modifications (PTM) thereof. For these reasons, various gene data banks such as NCBI, Uniprot, Ensemble, Expassy, and Brenda can be assessed and gone through to extract certain data.

In one experiment, biochemical pathways and the enzymes involved can be identified. The sequence of genes which produce the respective enzymes can be extracted. A significant amount of information about gene and protein structure can then be obtained from NCBI and Ensemble data banks. By using protein data banks such as Uniprot, Expassy, and similar resources, the proteins produced by these genes can be verified. This verification can include examination of the signal sequence and post-translational processes.

The upstream sequence of promoters can be identified as a harbor-safe sequence for gene introduction in that area. Analysis of this area can involve examining the NCBI and identifying 100-200 base pairs upstream of the promoter's region. Homology arms for donor templates can be designed from these regions. Considerations for this region (for introducing genes into the genome) can include minimizing conflict with the previous gene terminator and the upcoming promoter. Another factor to be considered in this region can be the identification of binding sites for certain proteins (e.g., transcription factors) in the area.

In order to identify the reaction of the Pichia cells to manipulate the genes (Knockout and Knock-in), certain systems biology software such as Optflex3 can be used. In this software, after the name of the microorganism is introduced and after the selection of the microorganism, it is possible to knock-out or knock-in certain genes and verify their effect on biomass value and growth.

The following aspects were considered to produce a KU70 knock-out strain in Pichia pastoris. CRISPR is a suitable technique for the process of gene knock-in and knock-out, and can be used in implementing the present technology. The CRISPR technique can employ two pathways—the first can be referred to as non-homologous end joining (NHEJ) and the second can be referred to as homologous recombination (HR). HR, in particular, can be used to knock-in the selected genes in the present technology.

In Pichia pastoris, unlike Saccharomyces cerevisiae, the process of HR is less utilized, where the Pichia strain was therefore manipulated to increase HR efficiency. The present technology therefore addresses the HR efficiency issue by silencing the Ku70 gene in Pichia. The Ku70 gene can be involved in NHEJ, and hence can be silenced to increase HR. A suitable microorganism for the HR processes described herein includes the GS115 strain of Pichia pastoris. The CRISPR technology can accordingly be used to knock-in several genes. At the outset, however, the Ku70 gene can be knocked-out in the GS115 Pichia pastoris genome to improve and enhance homologous recombination. For this purpose, GS115 can be modified to delete, disrupt, or otherwise silence the Ku70 gene. The GS115 strain provides a significant increase with respect to knock-in efficiency for genes of interest as it does not express the Ku70 gene. In the next stage, with the bioinformatics study on genes, the process of introducing some genes that interfere with the biosynthesis of cannabinoids can be considered.

The following aspects were used in CRISPR construct production (e.g., promotor-gene-terminator) in order to knock-in the designated genes into the Pichia pastoris genome. In order to increase the HR efficiency in the GS115 Pichia strain, the Ku70 gene involved in NHJE pathway is silenced. In the next stage, two plasmids are used to knock-in genes: BB3nK and BB3cH. A donor sequence can be provided in the BB3nK plasmid, where the gRNA and Cas9 can be provided in BB3cH plasmid. The BB3nK plasmid includes an ori sequence, an antibiotic resistance gene, and a donor or selected gene sequence. The BB3cH plasmid includes a hygromycin resistance gene, ori sequence, and an automated replication sequence (ARS), a Cas9 sequence, and a SgRNA sequence. gRNA designed by ChopChop Software and can be tested with other software, such as Cas-offinder. After designing the gRNA, it can be produced synthetically and/or by recombinant techniques for incorporation into the plasmid constructs.

For designing the donor template, a gene data bank can be used to extract the sequence including the coding sequence (CDS), where two homology arms for the donor template can be designed. The resulting sequence constructs can be assembled using various software, such as provided by Integrated DNA Technologies, Inc. (Coralville, Iowa, USA). Donor templates along with gRNA can also be constructed using synthetic and/or recombinant techniques. Finally, after the construction, the respective constructs can be cloned into the BB3nK and the BB3cH plasmids.

The following aspects can be used in Harbor Safe sequence development. Harbor Safe is a predetermined location of the genome in which to introduce the desired genes. Selecting this region for the CRISPR knock-in can allow gene expression and protein synthesis to be influenced by this area. Accordingly, certain bioinformatic characterization can be performed on this region. Suitable regions to use as a harbor-safe sequence to introduce genes into the genome can include locations about 100 base pairs upstream of certain gene promoters, for two reasons: (1) these regions are active for biological aspects, (2) there typically are no essential genes located in this space. Suitable upstream regions of the Pichia pastoris genes can be accordingly identified, and upstream regions of the AOX1, FLD1 and GAP promoter regions can be selected as harbor-safe sequences.

Construction of the SgRNA (ribozyme-gRNA-ribozyme) can include the following aspects. A sequence of twenty nucleotides can be prepared along with a sequence referred to as a scaffold, where the scaffold binds to the 3′ of a selected gRNA. Two ribozymes, including the hammerhead ribozyme (HH) and hepatitis delta virus ribozyme (HDR), can be connected to the two sides of the SgRNA, with the HH towards 5′ of the gRNA and the HDV towards the 3′ of the gRNA. Following transcription, the ribozymes function to cut two sides of the SgRNA (right and left), allowing assembly of the SgRNA.

Transfer of an assembled CRISPR construct into a cell by electroporation can include the following aspects. Electroporation techniques can be used to transfer the plasmid and construct (donor gRNA and CAS9) into the Pichia cells, where an electrical pulse can be used that leads to introducing the DNA into the cell. For this purpose, electrocompetent cells can be produced by using a condensed protocol that includes a short and quick method in comparison with regular electroporation. The donor template can be linearized prior to transforming the plasmid into the cell by electroporation. A restriction enzyme can be used to cut the donor template plasmid out of the donor sequence region. For example, the donor template plasmid can be cut with restriction enzyme to linearize the plasmid, after which the plasmid can be condensed and precipitated to produce a concentrated amount thereof (e.g., more than 1 ug per uL) by using absolute ethanol and sodium acetate (3M). Then 1.5 microgram donor plasmid with 100 nanograms CAS9 and gRNA plasmid can be transformed into the electrocompetent cells by electroporation. To produce electrocompetent Pichia cells, a BEDS solution (Bicine, Ethylene glycol, DMSO, and Sorbitol) can be used. A MicroPulser™, an electroporator from Bio-Rad (Hercules, California, USA), and a 2 mm cuvette with 1500 V and 25 uF can be used for the electroporation step. After electroporation, 1 ml of YPDS solution can be added and the cells can be incubated overnight at 30 degrees Celsius. The next day the samples can be cultivated on YPDS media selecting for hygromycin antibiotic resistance. After 72 hours, transformed colonies appeared.

The selection of transformants using selective media included the following aspects. Seventy-two hours following electroporation, the transformed colonies can be sorted from the media. The colonies can include three groups: (1) colonies that received the CAS9 and gRNA plasmid but the CAS9 cannot cut the DNA (due to the low efficiency of gRNA); (2) transformed colonies that received gRNA and CAS9 plasmid but the HR process was not completed and the donor sequence could not introduce into the genome, but the NHJE pathway repaired the cutting area; and (3) transformed colonies which received gRNA and CAS9 plasmid, as well as donor plasmid, went through the HR process, where this group is preferred and was subjected to verification. It was found to be better to select a small colony and have these colonies cultivated on YPD medium, where after a couple of generations, they gradually lose their CAS9 plasmid. Following 18-24 hours from cultivation on YPD medium, the transformed yeast can be plated by centrifugation and verified by DNA techniques.

PCR verification of transformants included the following aspects. Transformed colonies can be cultivated in a medium without antibiotics and after 18-24 hours DNA can be extracted from the colonies. PCR amplification can include gene-specific primer binding to the respective gene introduced to the genome. The primers can be designed with Primer 3 online software and the designed primers can be checked with Oligo Analyzer software. Suitable primers and primer pairs that minimize self-complementarity and dimers can be selected. It can be desirable to have the forward primer bind to the construct (e.g., the gene of interest) and the reverse primer bind to the Pichia pastoris genome to prevent the production of a false-positive. After designing the primers, the primers can be synthesized (e.g., by Integrated DNA Technologies) and PCR amplification can be performed using a Promega kit. In this way, the CRISPR construct and transformation efficiency can be optimized. After PCR amplification, the samples can be loaded onto an agarose gel and the presence of PCR product bands were visualized using a UV transilluminator.

RNA verification by Real-Time RT-PCR included the following aspects. After the transformed colonies containing the gene of interest are confirmed by PCR, the colonies can be verified at the RNA level to identify gene expression patterns. The samples including the genes of interest can be cultivated in a minimal medium called MGAs, which contain YNB, biotin, glycerol and ammonium sulphate. After 24 hours the yeast cells can be extracted by centrifuge and cultivated in an induction media including YNB, biotin, glycerol and methylamine (as a nitrogen source). The methylamine in induction media can induce the FLD1 promotor and lead to mRNA synthesis. RNA samples can be extracted using the Zymo research RNA kit in different time courses after induction (e.g., 0, 24 h, 48 h, 72 h, 96 h). cDNA can be synthesized from RNA using the cDNA synthesis Qiagen kit. Furthermore, Real-Time RT-PCR can be performed using the primers designed using primer 3 online software. The resulting amplicon can be expected to be 50 to 200 base pairs with the primers designed from the CDS of the gene.

Real-Time RT-PCR was done by using Qiagen QuantiNova Cyber green RT-PCR kit (Qiagen, Germantown, MD, USA), where GAPDH can be used as a positive internal control and GS115-KU70 can be used as a negative control. After sequentially performing RT-PCR for each sequential transformation of the five subject genes involved in CBGA biosynthesis, RT-PCR can be performed for all five of the genes in the final recombinant organism. Certain embodiments of the present technology produced interesting results. After induction with methylamine, the gene expression rate increased for five genes, and after 48 hours of induction the gene expression rate was suddenly reduced by a large degree. By increasing methylamine concentration, the cells can be under metabolic pressure (metabolic burden), therefore the transcription machinery can be under high pressure which subsequently leads to a gene expression decrease. To resolve this issue, the conditions can be optimized in order to decrease the metabolic burden. Three different strategies can be employed, including (1) decreasing the temperature to 15 degrees Celsius, (2) decreasing the inducer (methylamine) concentration, and (3) increasing the biomass in comparison to medium volume. These three strategies can be performed and samples obtained in different time courses, (e.g., 0, 24 h, 48 h, 72 h, and 96 h) to extract RNA for Real-Time RT-PCR amplification.

Protein verification by SDS-PAGE and western blotting techniques can include the following aspects. Once the genes are verified by PCR and the expression rate is identified by Real-Time RT-PCR, the expressed proteins can be validated by using the SDS-PAGE and western blotting technique. For this reason, certain protein tags (e.g., histidine tags) can be added to the 5′ or 3′ ends of the genes. Then, by using the anti-tag (e.g., anti-his tag) the desired protein can be identified using the western blot technique. The tag used in certain embodiments included a 6× histidine that can be introduced 3′ of the desired genes prior to stopping codons. Also, the polylinker sequence (e.g., Gly-Gly-Ser-Gly) can be used between the his-tag and gene sequence. In order to perform the SDS-PAGE and western blot, proteins can be extracted from different time courses after induction by using a yeast buster solution from Novagen. Protease inhibitors can be added to the yeast cell lysate to prevent protein degradation. Protein samples can be heated up to 100 degrees Celsius for five minutes and loaded on the polyacrylamide gels. Protein bands can be observed and transferred to the PVDF membrane by electroblotting and can be identified by using anti-his-tag bound to a colorimetric indicator. Suitable kits and corresponding protocols include those available from Nanoprobes Inc. (Yaphank, NY, USA).

Metabolite (e.g., substituted tryptamines) verification can include the following aspects. The metabolite production can be confirmed using an HPLC technique. In this study, the Hillic column (iHILIC® Fusion, 4.6×150 mm, 3.5 μm, 100 Å) can be used to extract the psychedelic compound using methanol. After extraction, the extraction samples can be injected into the specific columns and the results can be compared to psychedelic standard samples such as DMT and psilocin.

FIG. 1 depicts the biosynthesis pathways of certain substituted tryptamines, including psilocybin.

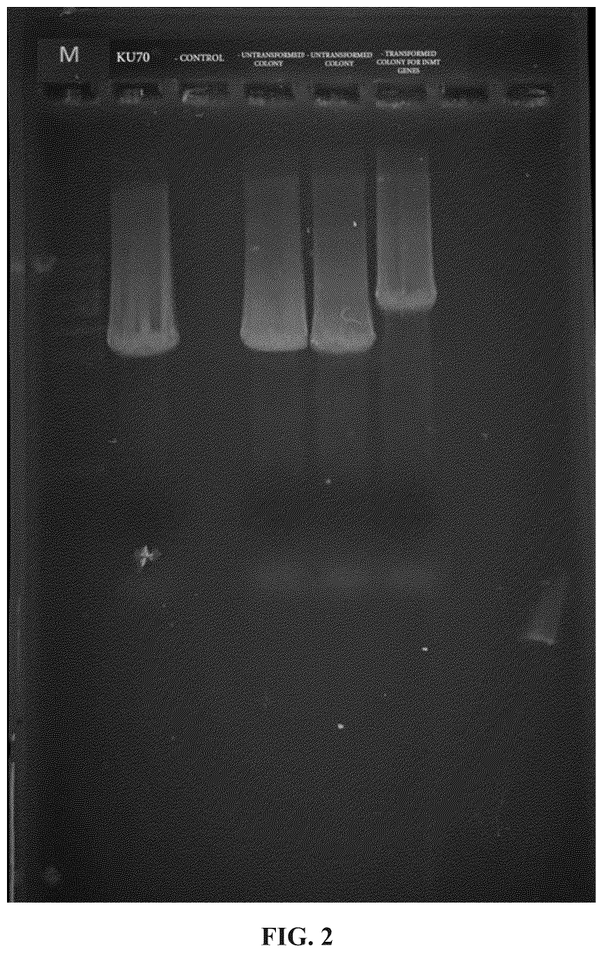

FIG. 2 depicts colony DNA verification for INMT genes. The colonies can be verified to select the transformed colony containing the INMT gene by providing two primers beside the homology arms (left and right homology arms). After amplification with the thermocycler using these primers, the PCR product can be loaded on the agarose gel (1%) and a difference can be seen between the transformed colony (heavy band) and the Ku70 (acting as a control that does not contain the INMT gene). As shown in FIG. 2, the Ku70 and the untransformed colonies can show smaller bands while the transformed colony can show a heavy band because the INMT gene can integrate into the Pichia genome, resulting in a heavy band.

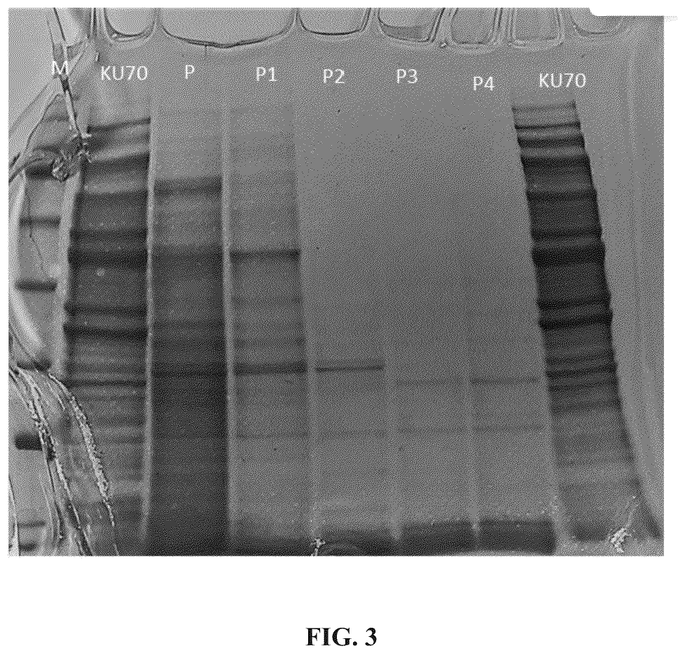

FIG. 3 depicts INMT protein verification in different time courses. FIG. 3 illustrates protein bands on polyacrylamide gel (12%). To see if the INMT gene is expressed or not, the protein can be extracted after the induction phase using promotor induction at different times. In each time, the total protein can be extracted from the culture. Finally, electrophoresis can be performed using the polyacrylamide gel. The band (30 KDa) illustrates the INMT protein, as expressed in the P3 and P4 times (not in the Ku70 or P-P1 and P2).

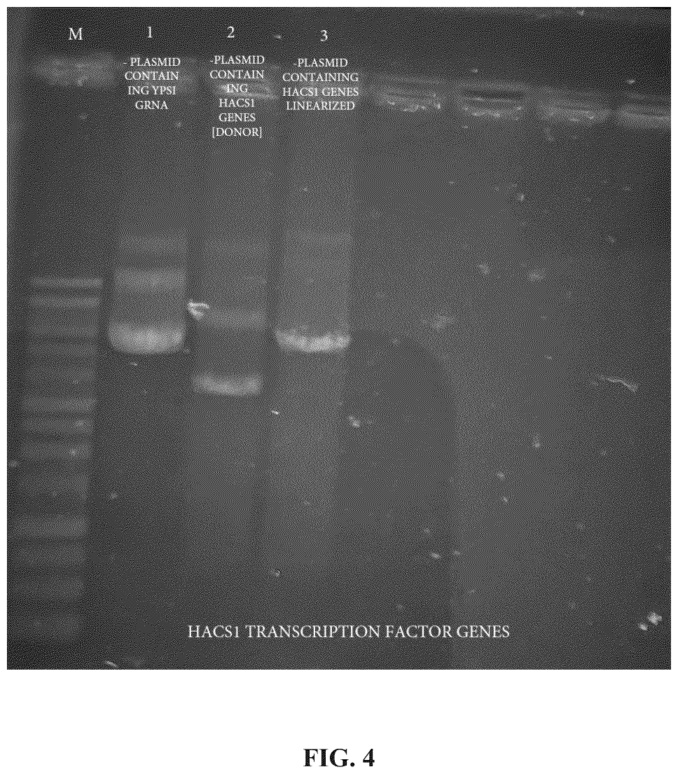

FIG. 4 depicts HACS1 (transcription factor) plasmid verification. FIG. 4 illustrates a plasmid band on agarose gel (1%). The plasmid can contain HRCS (transcription factor genes). Two different constructs can be used for the CRISPR experiment where one of the constructs can contain Co9 and SgRNA while the other construct can contain a donor template. The YPS plasmid on the left can contain Co9 and SgRNA, and as shown in FIG. 4, the second line is the donor template containing the HACS gene. The third line is the plasmid, which can be linearized for knock-in experiments.

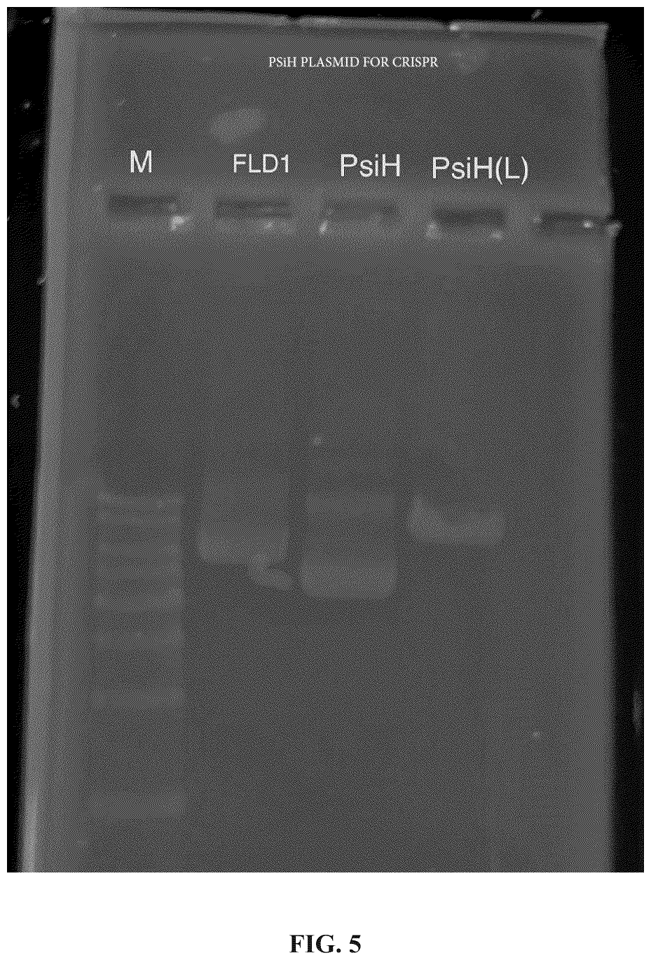

FIG. 5 depicts PsiH plasmids verification for CRISPR knock-in. FIG. 5 is similar to FIG. 4 but relates to the PSiH gene, FLD, and includes a construct containing Co9 and SgRNA.

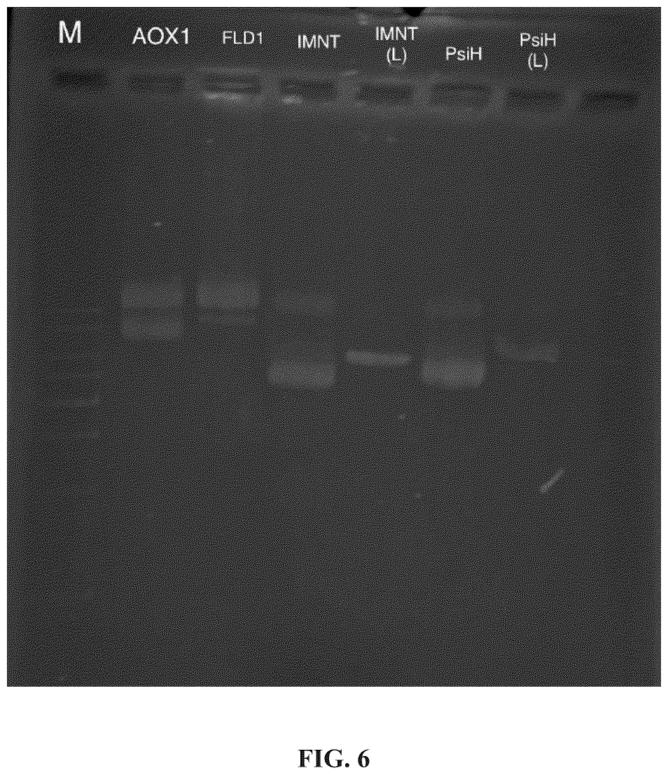

FIG. 6 depicts plasmid verification for INMT PsiH genes. Plasmid verification for INMT and PSiH genes, A0XI plasmid, contain cos9 and SgRNA for INMT genes, FLD, plasmid containing CoS9 and SgRNA for PSIH gene, plasmid which has (L) indicate linearization. For the linearization of INMT donor template, ECoRV restriction enzyme can be used and AfeI restriction enzyme can be used for PSiH template. The two primers used are designated as SEQ. ID. NO. 5 and SEQ. ID. NO. 6, which can also be used in reverse.

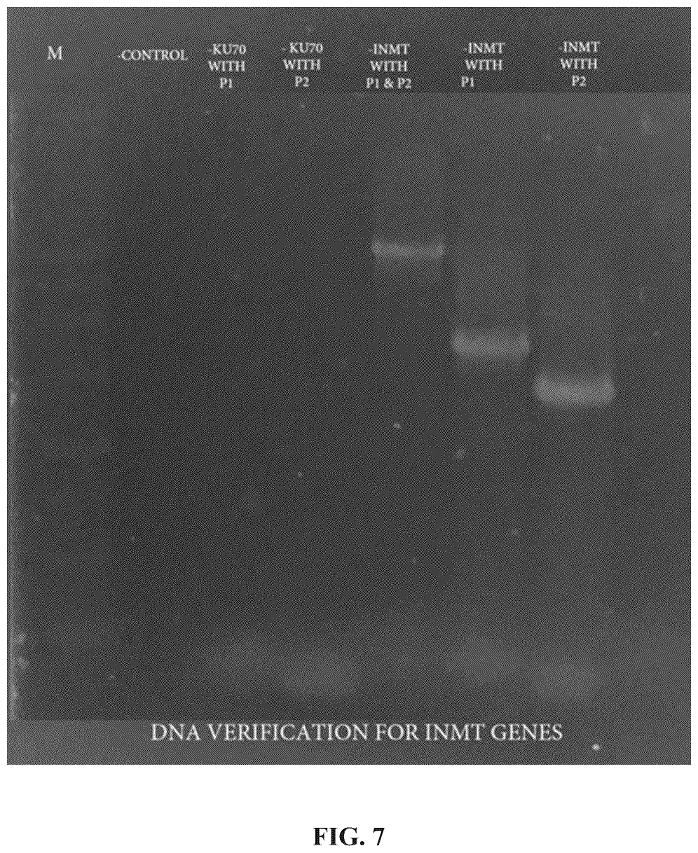

FIG. 7 depicts DNA verification for INMT genes. FIG. 7 illustrates DNA verification for the INMT gene with two different primers. As shown in FIG. 7, there is no band in the Ku70 line for both primers. These two primers (SEQ. ID. NO. 3 and SEQ. ID. NO. 4, which can also be used in reverse) can be designed from the sequence of the genome of Pichia and the sequence of the construct (INMT gene). After amplification with these primers, the PCR products can be loaded on the agarose gel 1% and UV stain can be used.

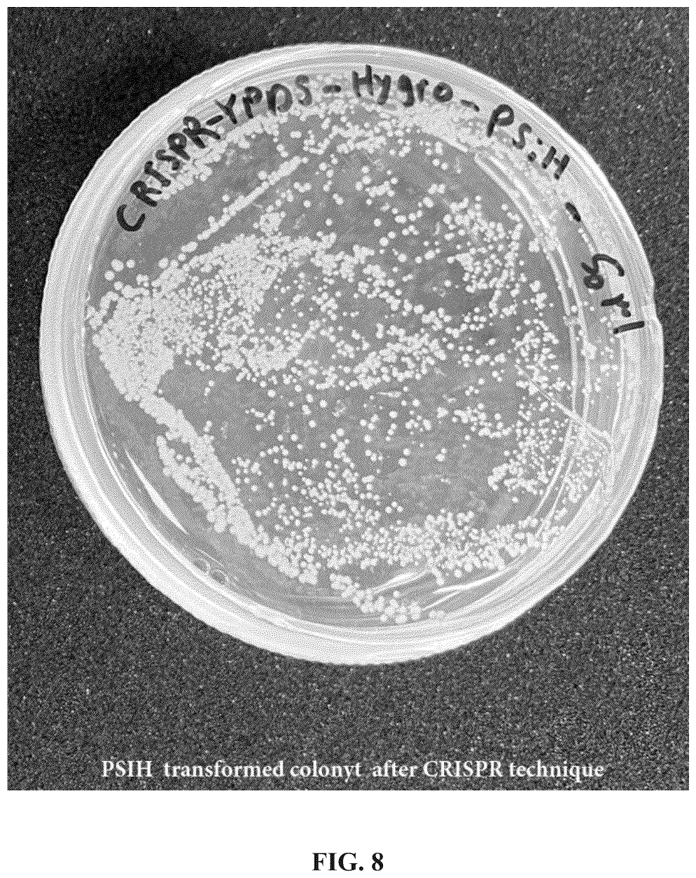

FIG. 8 depicts PsiH-transformed colonies after the CRISPR technique. As shown in FIG. 8, the colonies after CRISPR for PSiH knock-in experiments following electroporation of the two CRISPR constructs into the Pichia cells can be shown where the cells then cultured on the YPD+agar media containing hygromycin (200 fg/mL) to select the transformants.

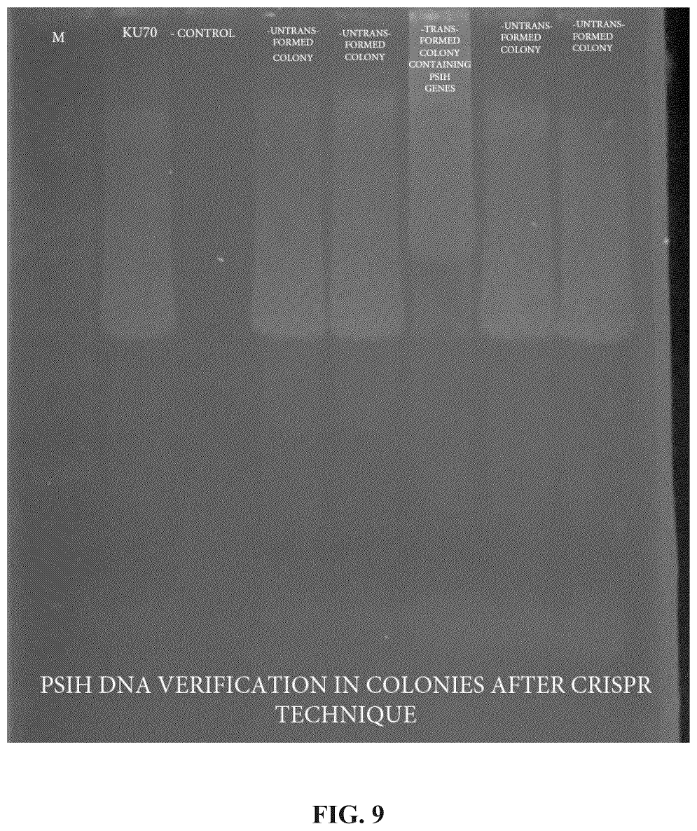

FIG. 9 depicts PsiH DNA verification in colonies after the CRISPR technique.

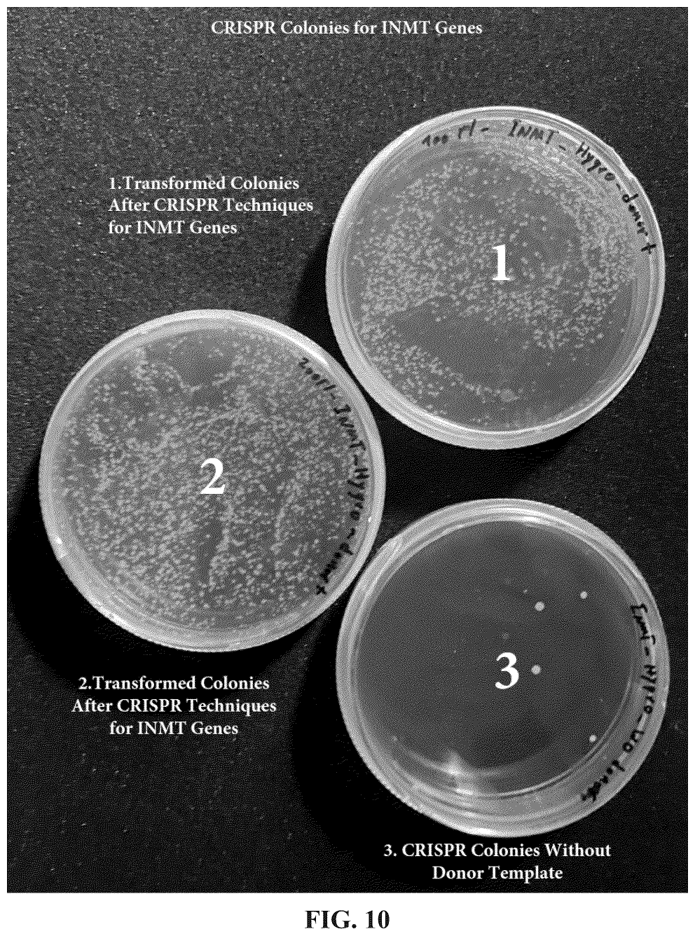

FIG. 10 depicts CRISPR colonies for INMT genes. FIG. 10 illustrates colonies after doing CRISPR technique. Number 3 can illustrate that if the donor template is absent because of breakage in the genome, the cells will be dead, as many colonies will not be seen. In numbers 1 and 2, the donor template can be transformed into the Pichia cell. Hence, the HDR system can integrate the donor to the genome by homologous recombination (HR), and the colonies can grow after 48 hours.

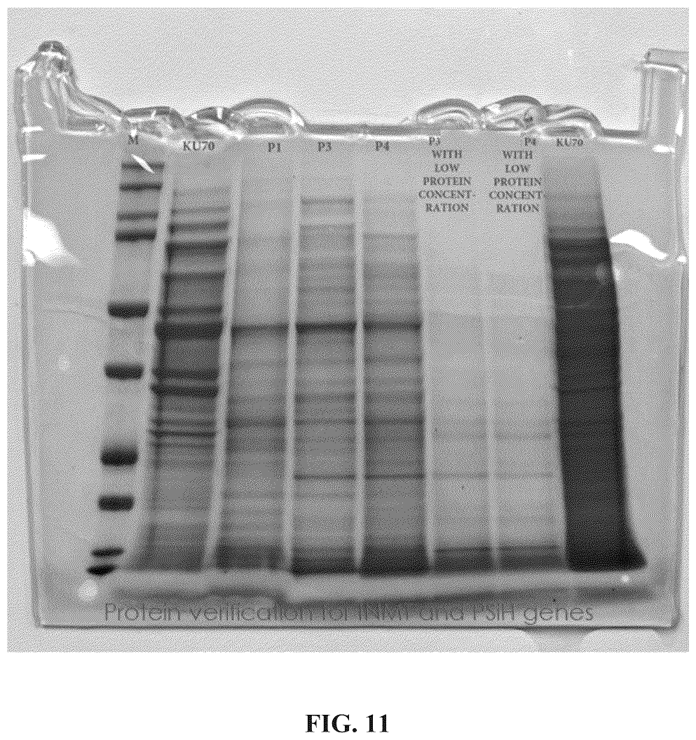

FIG. 11 depicts protein verification for INMT and PsiH genes. As shown in FIG. 11, the protein band can be seen, which indicates protein (enzyme) expression for 1 gene (INMT and PSiH) in different time courses after inductions with methanol.

Although use of a CRISPR construct is described to knock-in the selected genes into the Pichia genome, every CRISPR construct can have certain specific elements to introduce the construct into the genome. So, by changing the genes, some construct elements can be changed, which can also depend on the desired location in which to introduce the genes. One skilled in the art can select a predetermined location into which to introduce the genes and can ascertain the location, expression, and activity thereof in accordance with the present technology.

The enzymes provided herein, along with expression and activity thereof, can be readily characterized as described herein and understood by one skilled in the art. It should be appreciated that variants are contemplated by the present technology, where the expression and activity of such variants can be confirmed using the methods provided herein and understood by one skilled in the art. It is therefore possible to have nucleotide sequence variants of the subject genes and amino acid variants of the resultant proteins/enzymes based upon the degeneracy of the genetic code and/or based upon predetermined conservative amino acid substitutions that can be confirmed in accordance with the methods provided by the present disclosure to retain the desired expression level as well as enzymatic activity thereof. In this way, nucleotide sequence (and resultant amino acid sequence) can vary from the specific sequences listed herein. Embodiments include variants having from 75% up to 99.9% sequence identity to the specific sequences listed herein, where certain examples include sequence identities of 75%, 80%, 85%, 90%, 95%, 96%, 97%, 98%, and 99%.

Nucleotide or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned, and if necessary, gaps are introduced to achieve the maximum percent sequence identity. Sequence alignment procedures to determine percent identity are known to those skilled in the art. Publicly available computer software such as BLAST, BLAST2, ALIGN2 or Megalign (DNASTAR) software can be used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=(X/Y) (100), where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which an amino acid that is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell; e.g., P. pastoris.

The present technology allows production of substituted tryptamines, such as psilocybin, in a microorganism, such as Pichia pastoris. Production pathways can be tailored to produce various compounds related to psilocybin, such as one or more of 4-hydroxytryptamine, norbaeocystin (4-phosphoryloxy-tryptamine), NMT (N-methyltryptamine), 4-hydroxy-NMT (4-hydroxy-N-methyltryptamine), baeocystin (4-phosphoryloxy-N-methyl-tryptamine), DMT (N,N-dimethyltryptamine), psilocin (4-hydroxy-N,N-dimethyltryptamine), and the aforementioned psilocybin (4-phosphoryloxy-N,N-dimethyltryptamine).

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A recombinant organism configured to produce a substituted tryptamine, comprising:
a eukaryotic microorganism expressing a recombinant construct including an indolethylamine N-methyltransferase (INMT) and a dimethyl tryptamine 4-hydroxylase.

2. The recombinant organism of claim 1, wherein the eukaryotic organism includes a member of a fungus kingdom.

3. The recombinant organism of claim 1, wherein the eukaryotic organism includes a methylotrophic yeast.

4. The recombinant organism of claim 1, wherein the eukaryotic organism includes Pichia pastoris.

5. The recombinant organism of claim 1, wherein the indolethylamine N-methyltransferase (INMT) is derived from indolethylamine N-methyltransferase (INMT) expressed in human brain cortex.

6. The recombinant organism of claim 1, wherein the dimethyl tryptamine 4-hydroxylase is derived from dimethyl tryptamine 4-hydroxylase originating from Psilocybe cyanescens.

7. The recombinant organism of claim 1, wherein the recombinant construct is incorporated into the genomic DNA of the recombinant organism.

8. The recombinant microorganism of claim 1, wherein the recombinant construct is incorporated into the genomic DNA of the recombinant microorganism using CRISPR.

9. The recombinant microorganism of claim 8, wherein the CRISPR includes a homologous recombination technique.

10. The recombinant organism of claim 1, wherein the recombinant microorganism includes a KU70 gene knockout.

11. The recombinant microorganism of claim 10, wherein the KU70 gene knockout includes a selectable marker.

12. The recombinant microorganism of claim 1, wherein the recombinant construct is configured as a single recombinant element.

13. The recombinant microorganism of claim 1, wherein the recombinant construct is configured as multiple recombinant elements.

14. A recombinant microorganism configured to produce a substitute tryptamine, comprising:
a eukaryotic microorganism expressing a recombinant construct including an indolethylamine N-methyltransferase (INMT) and a dimethyl tryptamine 4-hydroxylase;
wherein:
the eukaryotic organism includes Pichia pastoris;
the indolethylamine N-methyltransferase (INMT) is derived from indolethylamine N-methyltransferase (INMT) expressed in human brain cortex;
the dimethyl tryptamine 4-hydroxylase is derived from dimethyl tryptamine 4-hydroxylase originating from Psilocybe cyanescens;
the recombinant construct is incorporated into the genomic DNA of the recombinant organism;
the recombinant construct is incorporated into the genomic DNA of the recombinant microorganism using CRISPR;
the recombinant microorganism includes a KU70 gene knockout.

15. A substituted tryptamine produced by a process comprising:
growing the recombinant microorganism configured to produce the substituted tryptamine of claim 1 in a growth medium; and
separating the substituted tryptamine from the recombinant microorganism and the growth medium.

16. A biosynthetic system for producing a substituted tryptamine, comprising:
a bioreactor;
the recombinant organism configured to produce the substituted tryptamine of claim 1; and
a growth medium for the recombinant microorganism.

17. A method of producing psilocin, comprising:
catalyzing the conversion of tryptamine to N-methyltryptamine using indolethylamine N-methyltransferase;
catalyzing the conversion of N-methyltryptamine to N,N-dimethyltryptamine using indolethylamine N-methyltransferase; and
catalyzing the conversion of N,N-dimethyltryptamine to psilocin using dimethyltryptamine 4-hydroxylase.

18. The method of claim 17, further including catalyzing the conversion of psilocin to psilocybin using a phosphotransferase enzyme.

19. The method of claim 17, further including extracting the psilocin using methanol.

20. A method of making psilocin, comprising:
growing the recombinant organism configured to produce the substituted tryptamine of claim 1 in a growth medium; and
separating the substituted tryptamine from the recombinant microorganism and the growth medium.
